(19) United States
(12) Patent Application Publication
(10) Pub. No.: US 2006/0058786 A1
(43) Pub. Date: Mar. 16, 2006

(54) Implant for correction of pectus excavatum

(75) Inventors: Kyung-Tae Kim, Seoul (KR); Jung-Sung Kim, Seoul (KR); Byung-Soo Kim, Seoul (KR); Tae-Jin Shin, Seoul (KR); Young-Ho Choi, Gyeonggi-do (KR)

Correspondence Address: MARGER JOHNSON & MCCOLLOM, P.C., 210 SW MORRISON STREET, SUITE 400, PORTLAND, OR, 97204, US

(73) Assignee: Medixalign Co., Ltd., Seoul (KR)

(21) Appl. No.: 10/529,032

(22) PCT Filed: Sep. 23, 2003
(86) PCT No.: PCT/KR03/01926

§ 371 Date: Mar. 23, 2005

(30) Foreign Application Priority Data
Sep. 28, 2002 (KR) 10-2002-0059059

(51) Int. Cl. A61B 17/56

(52) U.S. Cl. 606/60

(57) ABSTRACT

An implant for correcting pectus excavatum is disclosed, which comprises a chest correction bar (30) inserted into a body for lifting a depressed sternum and costal cartilages, and a stabilizer (40) for being inserted into a distal end of the chest correction bar (30) to prevent the chest correction bar from being rotated inside the body, wherein the chest correction bar (30) is formed at both jagged distal ends thereof with recesses (33) each of a predetermined length along the lengthwise direction of the chest correction bar (30), and wherein the stabilizer (40) comprises: two fixing plates (41, 41′) for being fixed to the body of a patient; a bridge (42) connecting the two fixing plates (41, 41′); two protruders (43, 43′) each generally opposed from the fixing plates (41, 41′) so as to be hitched by the recesses (33) at the distal ends of the chest correction bar (30) inserted from under the bridge (42), where there are formed two spaces (C) each of a predetermined size between the two protuders (43, 43′) and two lateral lengthwise surfaces of the bridge (42) so that the distal ends of the chest correction bar (30) can be inserted thereinto, thereby allowing the stabilizer (40) to be easily inserted into the chest correction bar (30), and once the insertion is made, pain and infection caused by stimulation on incised portions of a patient can be prevented.

### FIELD OF THE INVENTION

The present invention relates to an implant inserted into a body for correcting pectus excavatum.

### BACKGROUND OF THE INVENTION

In general, chest deformity is a case where a chest is more depressed or bulged than that of a normal person due to a depression or elevation of a sternum and surrounding costal cartilages. A depressed chest (pectus excavatum), also known as funnel chest, is particularly the most common anterior chest wall deformity for Asian people. The disfiguring physical appearance of this deformity can cause emotional and social impact especially among children, and may give rise to deterioration in growth or function of organs positioned near the chest area, such that doctors recommend that the depressed chest be operated in childhood.

One conventional surgical procedure for correcting pectus excavatum is to cut out a predetermined portion of inner costal cartilages positioned at both sides of a chest to form grooves therein. Sternum and costal cartilages are pulled forward about the grooves to form a proper thorax, and portions of the grooves at the costal cartilages are artificially filled in to correct the pectus excavatum.

However, there are many disadvantages in the conventional surgical procedure thus described in that the costal cartilages should be carved out from inside the chest, the sternum should be lifted and portions of the grooves must be filled in, thereby prolonging and complicating the operation procedure. It is also causes undue stress for both a surgeon and a patient who has to have his or her costal cartilages removed.

A surgical implant for performing the pectus excavatum procedure which does not suffer from the above-mentioned disadvantages is needed. One of these implants is disclosed in Korean Utility Model Registration No. 200581, which is hereby incorporated by reference, where an implant for lifting depressed sternum and costal cartilages is embedded into a body and fixed therein, thereby reducing the complexity of the surgical procedure, alleviating a patient's agony, and improving the cosmetic appearance of a person's chest.

The implant disclosed in the Korean Utility Model registration No. 200581 comprises a chest correction bar 10 for lifting the sternum and surrounding costal cartilages in the body, and a stabilizer 20 for being inserted into a distal end of the chest correction bar 10, as illustrated in FIG. 1. The chest correction bar 10 is formed at both distal ends thereof with a plurality of grooves 11 for hitching thread when the thread is sewn for fixing the chest correction bar 10 to a patient's body. The chest correction bar 10 is also formed at the furthest-most end thereof with a hole 12 for tying up the thread when the chest correction bar 10 is inserted into a body.

The stabilizer 20 is formed thereunder with an insertion piece 21 for inserting both ends of the chest correction bar 10 and is also formed with a fixation piece 22 of a predetermined length positioned at a right angle with the chest correction bar 10.

An operational procedure utilizing the conventional implant thus described is also disclosed in the Korean Utility Model registration No. 200581.

In particular, after a surgical tool fixed with a thread has penetrated the patient's chest from side to side, the thread is held by another tool while the surgical tool is pulled out after the implant has been imbedded, leaving the thread remaining in the chest. The thread is tied at the hole 12 formed at the furthest-most end of the chest correction bar 10. The thread is then pulled to allow the chest correction bar 10 to be fixed inside the body. When the chest correction bar 10 is inserted, a concave side thereof with a predetermined curvature should be in contact with the chest. Next, when both ends of the chest correction bar 10 are held and turned 180 degrees, the chest and costal cartilages are instantly lifted in accordance to the curved shape of the chest correction bar 10, forming the contour of the chest as desired. The chest correction bar 10 thus lifted is fixed using the grooves 11 at both ends thereof by being tied at the skin or muscle, and the stabilizer 20 is inserted into both ends of the chest correction bar 10 to prevent the chest correction bar 10 from being rotated.

There is a disadvantage in the implant for correcting pectus excavatum thus described according to the prior art in that, because a planar surface of the fixation piece 22 at the stabilizer 20 is protrusively formed with the insertion piece 21, the overall thickness of the stabilizer 20 becomes larger, such that when the chest correction bar 10 is inserted, soft tissue around the operated portion are stimulated, causing pain to a patient, and in worst cases, soft tissue may become infected.

Still worse, it is difficult to insert the stabilizer 20 to the body-fitted chest correction bar 10 through a small incised portion because the fixation piece 22 should be inserted in the parallel state with a planar surface of the chest correction bar 10 when the stabilizer 20 is inserted into the chest correction bar 10.

### SUMMARY OF THE INVENTION

The present invention provides an implant for correction of pectus excavatum in which a stabilizer is easily inserted into a chest correction bar. Once the stabilizer is inserted, pain and infection caused by stimulation to incised portions of a patient can be prevented.

The implant for correction of pectus excavatum according to the present invention comprises a chest correction bar going through a body for lifting a depressed sternum and costal cartilages, and a stabilizer for being inserted into a distal end of the chest correction bar to prevent the chest correction bar from being rotated inside the body, wherein the chest correction bar is formed at both jagged distal ends thereof with recesses each of a predetermined length along the lengthwise direction of the chest correction bar. The stabilizer comprises two fixing plates for being fixed to a body of a patient, a bridge connecting the two fixing plates, and two protruders each generally opposed from the fixing plates so as to be hitched by the recesses at the distal ends of the chest correction bar inserted from under the bridge, where, between the two protruders and two lateral lengthwise surfaces of the bridge, there are formed two spaces each of a predetermined size so that the distal ends of the chest correction bar can be inserted thereinto.

Preferably, the protruders are pins attached to the fixing plates.

The fixing plates are formed at lateral surfaces thereof with grooves at which threads can be hitched when the threads are tied for securing the stabilizer to the body of a patient.

Preferably, the fixing plates are centrally formed with through holes for reducing the weight of the stabilizer and for hitching threads as well in case of need.

Preferably, the central planar portion of the chest correction bar is cut out lengthwise such that the thickness of the central portion of the chest correction bar is thinner than that of the distal ends thereof.

### BRIEF DESCRIPTION OF THE DRAWINGS

For fuller understanding of the nature and objects of the present invention, reference should be made to the following detailed description taken in conjunction with the accompanying drawings in which:

FIG. 1 is an exploded perspective view of an implant for correcting pectus excavatum according to the prior art;

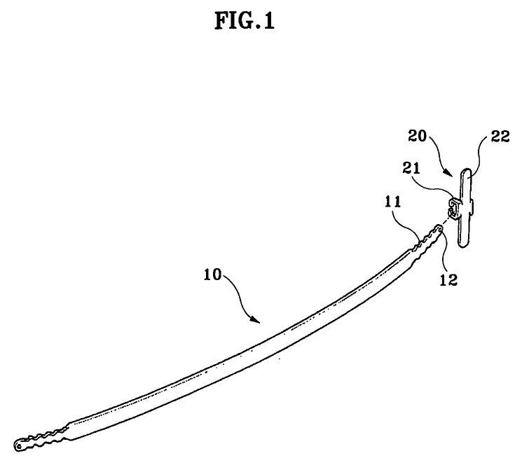

FIG. 2 is a coupled perspective view of an implant for correcting pectus excavatum according to the first embodiment of the present invention;

FIG. 3 is a partial cross-sectional view taken along A-A of FIG. 2, where only cross-sections of both sides of the stabilizer and cross-section of the chest correction bar are shown;

FIGS. 4a and 4b are perspective views of the chest correction bar and the stabilizer for implant according to the first embodiment of the present invention;

FIG. 5 is plan view of the stabilizer of FIG. 4b;

FIGS. 6-8 are constitutional views where a stabilizer is inserted into distal ends of a chest correction bar for implant according to the first embodiment of the present invention;

FIG. 9 is a perspective view of a stabilizer for implant according to a second embodiment of the present invention; and

FIG. 10 is a perspective view of a chest correction bar for implant according to a third embodiment of the present invention.

### DETAILED DESCRIPTION OF THE PREFERRED EMBODIMENTS

The preferred embodiments of the present invention will now be described in detail with reference to the accompanying drawings.

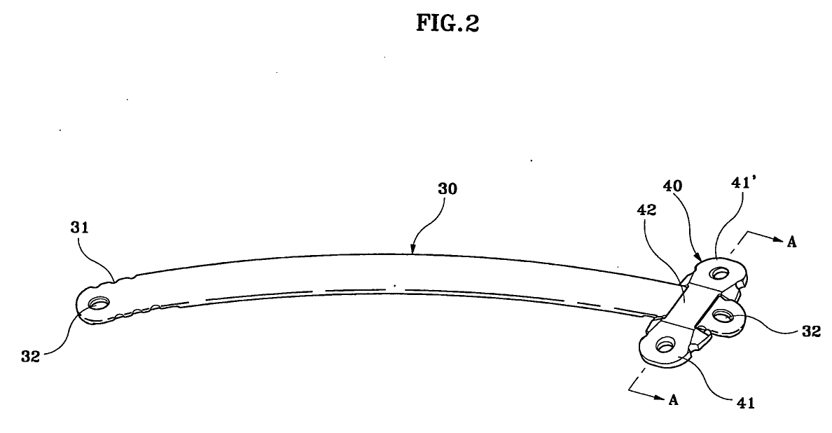

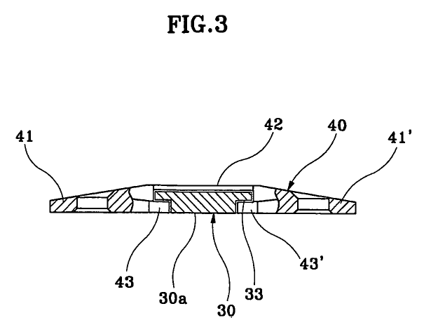

FIG. 2 is a coupled perspective view of an implant for correcting pectus excavatum according to the first embodiment of the present invention and FIG. 3 is a partial cross-sectional view taken along A-A of FIG. 2.

As depicted in the drawings, the implant according to the present invention comprises a chest correction bar 30 going through a body for lifting a depressed sternum and surrounding costal cartilages, and a stabilizer 40 for being inserted into a distal end of the chest correction bar 30 to prevent the chest correction bar 30 from being rotated inside the body.

The chest correction bar 30 and the stabilizer 40 are made of unharmful and rust-proof biocompatible metals such as stainless steel, titanium alloy, cobalt-chrome alloy and the like, and also may be made of biocompatible polymer or copolymer such as Utra High Molecular Weight Polythylene (UHMWPE), Poly L-Lactide Acid (PLLA), Poly Glycolic Acid (PGA), Poly D-Lactide Acid (PDLA).

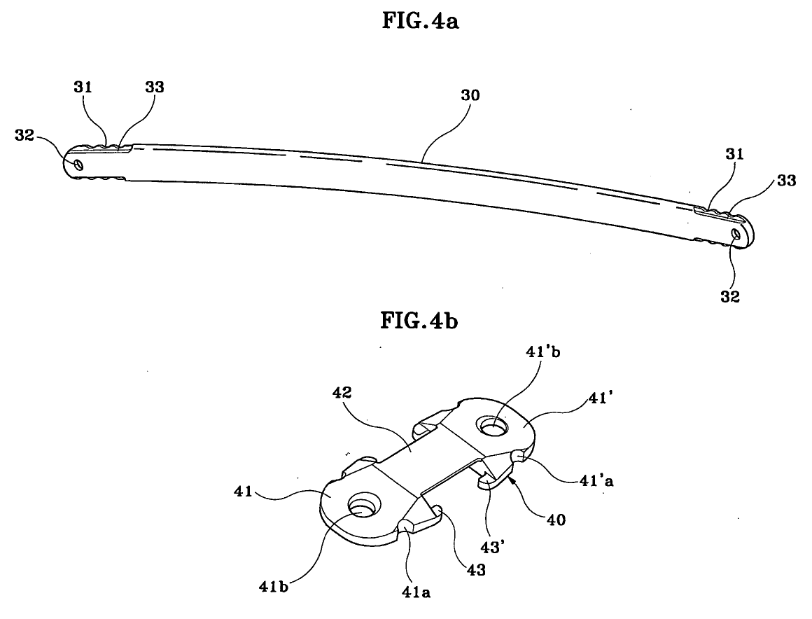

As shown in FIGS. 2, 4a and 4b, the chest correction bar 30 features a curved strip-type elongated bar having a predetermined curvature to smoothly connect costal cartilages at both sides of a body and to lift the sternum and the costal cartilages, and has a bending strength and stiffness so that the curvature of the chest correction bar 30 can be appropriately adjusted in relation to the chest width and chest contour of a patient.

The chest correction bar 30 has a planar surface. Although it is preferred that the bar 30 is bent for use by a patient according to his or her chest contour, it is also possible that the bar 30 is manufactured with a predetermined contour. In the first embodiment of the present invention, the bar 30 is bent with an arbitrary contour.

The chest correction bar 30 is formed at both marginal end surfaces thereof with a plurality of grooves 31 so as to be hitched when threads are tied for securing the bar 30 to the body of a patient.

The chest correction bar 30 is also formed at both furthestmost distal ends thereof with through holes 32 for holding threads when the bar 30 is inserted into a body. Furthermore, inwardly bent sides of both distal ends of the chest correction bar 30 are lengthwise formed with recesses 33 each of a predetermined length.

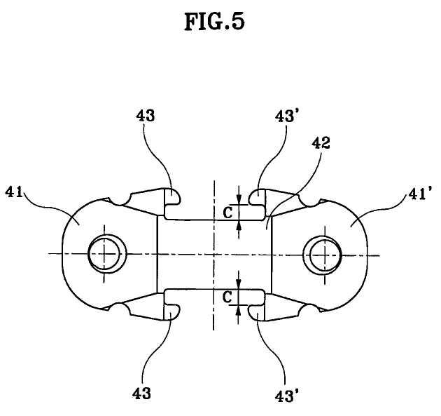

The stabilizer 40 comprises: two fixing plates 41 and 41′ for being fixed to the body of a patient; a bridge 42 connecting the two fixing plates 41 and 41′; two protruders 43 and 43′ each generally opposed from the fixing plates so as to be hitched by the recesses 33 at the distal ends of the chest correction bar 30 inserted from under the bridge 42, where, between the two protruders 43 and 43′ and two lateral lengthwise surfaces of the bridge 42, there are formed two spaces (C) each of a predetermined size so that the distal ends of the chest correction bar 30 can be inserted thereinto (refer to FIG. 5).

The fixing plates 41 and 41′ are formed at lateral surfaces thereof with lateral grooves 41a and 41′a for holding thread when the thread is tied for securing the stabilizer 40. The fixing plates 41 and 41′ are centrally formed with through holes 41b and 41′b for reducing the weight of the stabilizer 40 and for holding the thread in case of need.

The operating method of using the above-identified implant thus described according to the present invention in which the implant is inserted into the body of a patient and tied by thread is the same as that of the prior art.

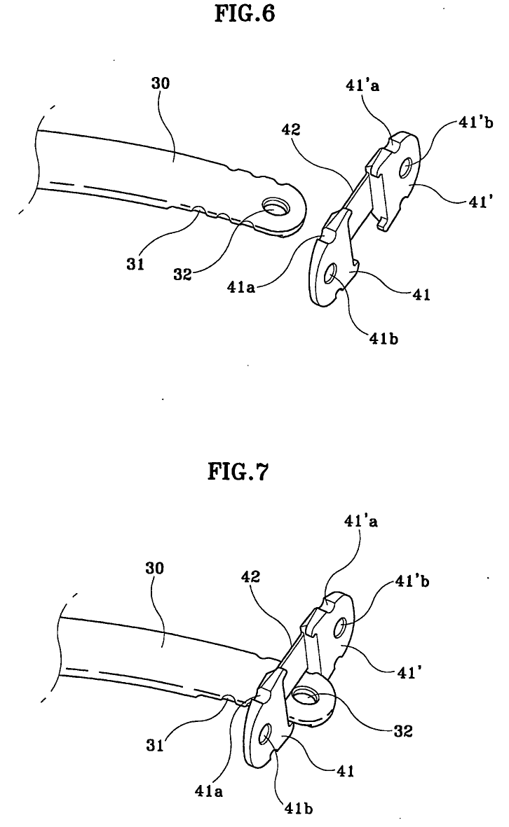

Furthermore, distal ends of the chest correction bar 30 inserted into the body of a patient and protruding out of the body at both ends thereof are fitted by a stabilizer. As illustrated in FIG. 6, the planar surface of the stabilizer 40 is disposed at a right angle by planar surface of the chest correction bar 30, which in turn is inserted into the spaces depicted as C (refer to FIG. 5) formed by the protruders 43, 43′ and widthwise lateral surfaces of the bridge 42 of the stabilizer 40 as shown in FIG. 7. Then the stabilizer 40 is rotated as seen in FIG. 8 to allow both planar surfaces of the stabilizer 40 and the chest correction bar 30 to be in parallel, and the stabilizer 40 is insertedly coupled in the lengthwise direction of the chest correction bar 30. As a result, the stabilizer 40 can be easily inserted into the chest correction bar 30 that is closely contacting the body.

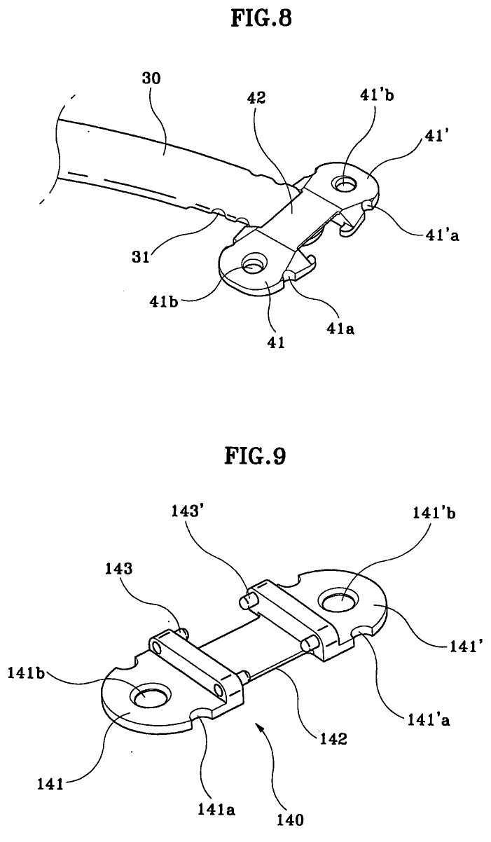

FIG. 9 is a perspective view of a stabilizer of an implant according to a second embodiment of the present invention.

The stabilizer 40 according to the teachings of the second preferred embodiment of the present invention is mounted with the protruders of the first embodiment in the form of pins 143 and 143′ attached to fixing plates 141 and 141′. The bridge 142, lateral grooves 141a and 141′a and through holes 141b and 141′b are the same as those of the first embodiment.

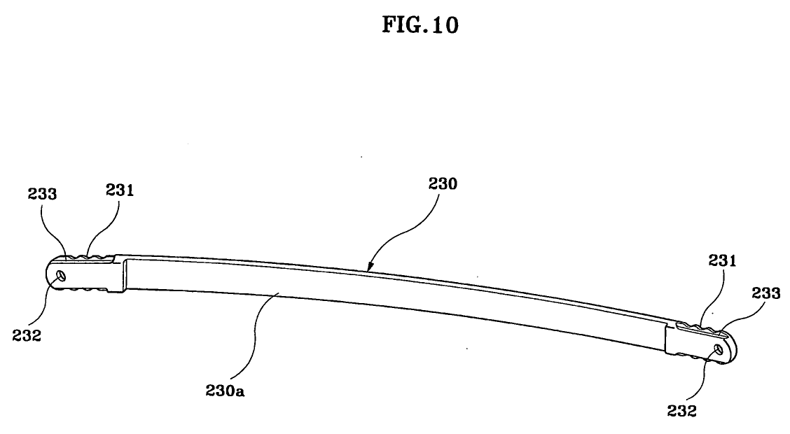

FIG. 10 is a perspective view of a chest correction bar of an implant according to a third embodiment of the present invention.

An intermediate thickness assigned to a chest correction bar 230 according to the teachings of the third preferred embodiment of the present invention is thinner than distal ends of the chest correction bar such that a central portion of the chest correction bar 230 in between the two distal ends thereof is hollowed. Construction of lateral grooves 231, through holes 232 and recesses 233 are the same as that of the first embodiment of the present invention.

The chest correction bar 230 of the teachings of the third embodiment of the present invention therefore may be reduced in weight due to the hollowed central portion thereof to be stably coupled with a stabilizer.

The foregoing discussion has disclosed and described merely exemplary embodiments of the present invention. It is not intended to be exhaustive or to limit the invention to the precise form disclosed, and modifications and variations are possible in light of the above teachings or may be acquired from practice of the invention.

As apparent from the foregoing, there is an advantage in the implant for correcting pectus excavatum thus described according to the present invention in that it is easy to insert a stabilizer to a chest correction bar due to the thinness of the stabilizer, and once the stabilizer is inserted, pain and infection caused by stimulation to incised parts of a patient can be prevented.

There is another advantage in that, when the stabilizer is inserted to the chest correction bar, the planar surface of the stabilizer is initially inserted at right angle into the planar surface of the chest correction bar but later rotated to place itself in parallel position with the planar surface of the chest correction bar, making it easy to insert the stabilizer.

What is claimed is:

1. An implant for correcting pectus excavatum, comprising:
a chest correction bar for lifting a depressed sternum and costal cartilages; and
a stabilizer for being inserted into a distal end of said chest correction bar to prevent said chest correction bar from being rotated inside the body,
wherein said chest correction bar is formed at both distal ends thereof with recesses along the lengthwise direction of said chest correction bar, and wherein said stabilizer comprises: two fixing plates for being fixed to the body of a patient; a bridge connecting the two fixing plates; two protruders each opposed from the fixing plates so as to be hitched by said recesses of said chest correction bar inserted from under the bridge, where there are formed two spaces between said two protruders and said two lateral widthwise surfaces of the bridge, each space being of a predetermined size so that the distal ends of said chest correction bar can be inserted thereinto.

2. The implant as defined in claim 1, wherein said protruders are composed of pins attached to the fixing plates.

3. The implant as defined in claim 1, wherein said fixing plates are formed at lateral surfaces thereof with grooves for hitching thread when the thread is tied for securing said stabilizer to the body of a patient.

4. The implant as defined in claim 1, wherein said fixing plates are centrally formed with through holes for reducing the weight of said stabilizer and for hitching thread in case of need.

5. The implant as defined in claim 1, wherein the central planar portion of said chest correction bar is cut out lengthwise such that thickness of the central portion of said chest correction bar is thinner than that of the distal ends thereof.
